(19) United States
(12) Patent Application Publication
(10) Pub. No.: US 2024/0054568 A1
(43) Pub. Date: Feb. 15, 2024

(54) COMPUTER-IMPLEMENTED METHOD OF MANAGING AN INSURANCE CLAIM

(71) Applicant: PETSURE (AUSTRALIA) PTY LTD, Chatswood (AU)

(72) Inventors: Toby Yoo, Chatswood (AU); Geoffrey Nigel Mathers, Chatswood (AU)

(21) Appl. No.: 18/243,415

(22) Filed: Sep. 7, 2023

Related U.S. Application Data
(63) Continuation-in-part of application No. 17/280,696, filed on Mar. 26, 2021.

(30) Foreign Application Priority Data
Sep. 28, 2018 (AU) 2018903667

(51) Int. Cl. G06Q 40/08; G16H 10/60

(52) U.S. Cl. CPC G06Q 40/08; G16H 10/60

(57) ABSTRACT

The present invention is broadly directed to a computer-implemented method of managing an insurance claim via a computer system and an associated software platform 10 which performs the steps of:
1. receiving claim data at 12a to 12c associated with an insurance claim made under an insurance policy of an insurance provider 13, the insurance claim being made in respect of treatment of an animal at a veterinary practice 14;
2. processing the claim data at 16 to provide a claim workflow derived from the claim data 12a;
3. electronically submitting an integrated claim workflow to the insurance provider 13 for assessment of the insurance claim at 18 under the insurance policy;
4. receiving notification at 20 from the insurance provider 13 of a benefit amount to be paid by the insurance provider 13 under the insurance policy for the treatment;
5. electronically informing the veterinary practice 14 of the benefit amount to be paid by the insurance provider 13 to either the veterinary practice 14 or a policy owner of the insurance policy.

### TECHNICAL FIELD

The present invention relates broadly to a computer-implemented method of managing an insurance claim for treatment of an animal. The invention is also broadly directed to a computer system for managing an animal insurance claim.

### BACKGROUND

Trupanion Inc. in their US patent publication no. 2017/0039329 describe a pet insurance system and method. More particularly, Trupanion describe an architecture where they supply an “agent” (computer program) which is capable of interacting with the software at a veterinary practice. The veterinary practice submits an insurance claim for a pet treatment on behalf of the pet owner or other customer from a client/browser application or website referred to as “Trupanion Express”. The Trupanion Express” website uses the “agent” program and data entered by the veterinary practice to submit the insurance claim to Trupanion servers. The veterinary practice enters the relevant data for the pet treatment via the Trupanion website. The exchange of information between the veterinary practice and Trupanion is in a proprietary data format. Trupanion quickly adjudicates the insurance claim and, on approval of the claim, Trupanion directly pays the veterinary practice for the treatment in near real time. The pet owner then pays for their portion of the treatment to the veterinary practice and Trupanion are notified of this payment via the “Trupanion Express” website.

### SUMMARY OF INVENTION

According to a first aspect of the present invention there is provided a computer-implemented method of managing an insurance claim, said method comprising the steps of:
receiving claim data associated with an insurance claim made under an insurance policy of an insurance provider, said insurance claim being made in respect of treatment of an animal at a veterinary practice;
processing the claim data to provide a claim workflow derived from said claim data;
electronically submitting an integrated claim workflow to the insurance provider for assessment of the insurance claim under the insurance policy, the integrated claim workflow being derived from the claim workflow and provided in a format compatible for submission to the insurance provider;
receiving notification from the insurance provider of a benefit amount to be paid by the insurance provider under the insurance policy for said treatment;
electronically informing the veterinary practice of the benefit amount to be paid by the insurance provider to either the veterinary practice or a policy holder of the insurance policy.

Preferably the method also comprises the step of receiving notification of payment of a remaining amount from the policy holder or an associated individual to the veterinary practice for the treatment of the animal, the remaining amount calculated by deducting the benefit amount from an invoiced amount for said treatment. More preferably the notification of payment of the remaining amount is transmitted to the insurance provider prompting payment by the insurance provider of the benefit amount to the veterinary practice or the policy owner. Still more preferably the remaining amount is paid at the veterinary practice the policy owner or the associated individual at the time of attending the veterinary practice for the treatment.

Preferably the method further comprises the step of reading the claim data to identify an insurance claim as a benefit claim where the veterinary practice has categorised the insurance claim as potentially being eligible for payment of a benefit amount by the insurance provider. More preferably, in the event the insurance claim is identified as a benefit claim, the method further comprises the step of prompting the insurance provider to assess the insurance claim following its receipt and processing. Even more preferably said prompting involves automatically sending a telecommunication alert to the insurance provider notifying them of the receipt of the insurance claim and in anticipation of submission of the integrated claim workflow associated with the insurance claim.

Preferably the step of receiving notification of the benefit amount from the insurance provider involves electronically querying the insurance provider in relation to a benefit amount being payable for the treatment of the animal at the veterinary practice. More preferably said step of querying the insurance provider involves periodic polling of the insurance provider in relation to the benefit amount. Even more preferably in the event of a change in the status of the insurance claim, the step of receiving notification of the benefit amount also involves the insurance provider responding to the periodic polling with notification of the benefit amount.

Preferably the method also comprises the step of storing the notification of the benefit amount received from the insurance provider in an insurance integration database from which the integrated claim workflow is electronically submitted. More preferably the notification of the benefit amount is entered into the insurance integration database for storage via an associated insurance user interface.

Preferably the method still further comprises the step of sending the notification of the benefit amount from the insurance integration database to a claim workflow database associated with the claim workflow. More preferably the notification of the benefit amount is converted to a compatible data format capable of being read by the veterinary practice. Still more preferably the compatible data format is based on a canonical data model. Even still more preferably the notification of the benefit amount in the compatible data format is stored in the claim workflow database.

Preferably the step of electronically informing the veterinary practice of the benefit amount involves prompted delivery of the notification of the benefit amount to the veterinary practice in the compatible data format in response to periodic polling from the veterinary practice. Alternatively the benefit amount in the compatible data format is delivered to the veterinary practice by calling the veterinary practice for pushing of the benefit amount to said practice.

According to a second aspect of the invention there is provided a computer system for managing an animal insurance claim, said system comprising:
a storage component including a claim workflow database for storing a claim workflow derived from claim data associated with an insurance claim for treatment of an animal;
a submitting component arranged to communicate with the storage component, said submitting component being configured to electronically submit an integrated claim workflow to an insurance provider for assessment of the insurance claim under an insurance policy pertaining to the animal, the integrated claim workflow being derived from the claim workflow and provided in a format compatible for submission to the insurance provider;
a notification component configured to receive a notification from the insurance provider of a benefit amount to be paid by the insurance provider under the insurance policy for said treatment;
an informing component arranged to communicate with the notification component, said informing component being configured to electronically inform the veterinary practice of the benefit amount to be paid by the insurance provider to either the veterinary practice or a policy owner of the insurance policy.

According to a third aspect of the invention there is provided a computer system for managing an animal insurance claim, said system comprising:
a processor;
a memory device which stores a plurality of instructions which when executed by the processor cause the processor to:
   receive claim data associated with an insurance claim made under an insurance policy of an insurance provider, said insurance claim being made in respect of treatment of an animal at a veterinary practice;
   process the claim data to provide a claim workflow derived from said claim data;
   electronically submit an integrated claim workflow to the insurance provider for assessment of the insurance claim under the insurance policy, the integrated claim workflow being derived from the claim workflow and provided in a format compatible for submission to the insurance provider;
   receive notification from the insurance provider of a benefit amount to be paid by the insurance provider under the insurance policy for said treatment;
   electronically inform the veterinary practice of the benefit amount to be paid by the insurance provider to either the veterinary practice or a policy owner of the insurance policy.

### BRIEF DESCRIPTION OF DRAWINGS

In order to achieve a better understanding of the nature of the present invention a preferred embodiment of a computer-implemented method of managing an insurance claim for treatment of animal will now be described, by way of example only, with reference to the accompanying illustrations in which:

FIG. 1 is a schematic flow diagram showing data flow between the various components of a computer system including a software platform for managing an animal insurance claim according to one aspect of the invention;

FIG. 2 is a schematic flow diagram of a method of managing an insurance claim according to another aspect of the invention illustrating implementation of part of the method via the software platform of FIG. 1 built in a microservice architecture;

FIG. 3 is a schematic flow diagram of the remainder of the method of the embodiment of FIG. 2 showing implementation of this aspect of the invention via the software platform of FIG. 2;

FIGS. 4A to 4C are schematic block diagrams depicting workflows and data flow from the computer system and the method of the preceding figures; and

FIGS. 5A and 5B show partial claim data in XML format illustrating claim data augmentation;

FIG. 5C is an exemplary flow chart of the described method of the software platform in the context of the claim augmentation of FIGS. 5A and 5B;

FIGS. 6A and 6B are exemplary flow charts of the described systems and processes of the software platform of both aspects of the technology.

### DETAILED DESCRIPTION

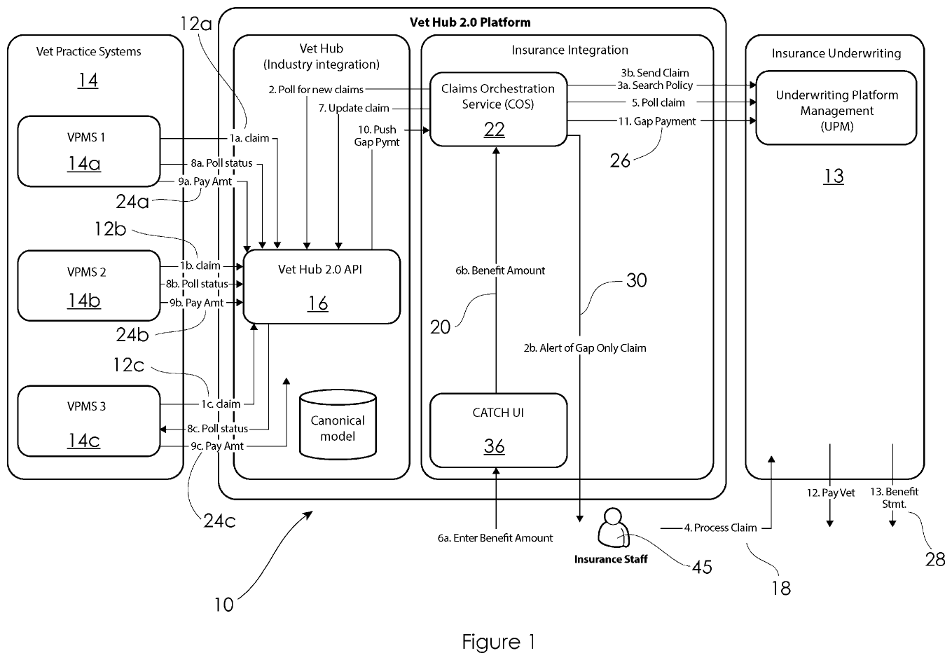

As best seen in FIG. 1, there is a computer-implemented method of managing an insurance claim according to one aspect of the invention. The method of this embodiment is enabled via a computer system and an associated software platform 10 which broadly performs the steps of:
1. receiving claim data at 12a to 12c associated with an insurance claim made under an insurance policy of an insurance provider 13, the insurance claim being made in respect of treatment of an animal at a veterinary practice 14;
2. processing the claim data at 16 to provide a claim workflow derived from the claim data such as 12a;
3. electronically submitting an integrated claim workflow to the insurance provider 13 for assessment of the insurance claim at 18 under the insurance policy;
4. receiving notification at 20 from the insurance provider 13 of a benefit amount to be paid by the insurance provider 13 under the insurance policy for the treatment;
5. electronically informing the veterinary practice 14 of the benefit amount to be paid by the insurance provider 13 to either the veterinary practice 14 or a policy owner of the insurance policy.

In this embodiment the integrated claim workflow is derived from the claim workflow and provided in a format compatible for submission to the insurance provider 13. The integrated claim workflow is constructed at the claims orchestration service (COS) at 22. The COS 22 is modular in the sense that it is tailored to align the integrated claim workflow and associated data with the insurance provider or more particularly the underwriting platform to the UPM 13 by translating the claim workflow of the associated application program interface (API) into an integrated claim workflow which is compatible with the selected UPM 13. Each veterinary practice 14 of different veterinary practices in making an insurance claim can rely upon its own different software and associated user interface to communicate directly with the UPM 13. To this end, the respective user interface of different veterinary practices may be configured to display a predetermined set of common indicators. Because the indicators are common across the configured user interfaces of the different veterinary practices, the veterinary practices can provide and transmit common insurance information (e.g. policy number, policy owner indicator, patient/pet name etc) expected by the insurance providers. Further, the respective user interface may be configured to display information received from the insurance provider. The information received from the insurance provider may be provided in one or more forms, such as via the software or associated user interface at the veterinary practice 14 or via email. For example, the information received may include reconciliation data associated with one or more past benefit payment amounts. The respective user interface may display reconciliation indications to indicate the reconciliation data. The reconciliation data may be useful for veterinary practices to reconcile a list of benefit amounts indicated to be received against benefit amounts actually received from the insurance provider. For each benefit payment (compensation), the reconciliation data may include any one or more of: a fee for the transmission of the compensation, an effective compensation based on the compensation reduced by the fee; a date of the transmission of the compensation; and a transmission reference for the transmission of the compensation. At least some of the reconciliation data may be received from the insurance provider on a regular basis, such as daily and/or monthly. The respective user interface may be configured to display at least some, and in some cases not all, of the reconciliation data. For example, the respective user interface may be configured to display the benefit amount per claim and omit any display of the fee.

In this example the method also comprises the step of receiving notification of payment of a remaining or gap amount at 24a to 24c made by a policy owner or an associated individual at the time of attending the veterinary practice 14 for the treatment. It will be understood that the remaining amount which is colloquially referred to as the gap is calculated by deducting the benefit amount to be paid by the insurance provider from an invoiced amount for the treatment by the veterinary practice 14. In this case the notification of payment of the gap is transmitted to the insurance provider at 26 prompting payment by the insurance provider 13 of the benefit amount at 28 to the veterinary practice 14. If the policy owner elects to pay the invoiced amount in full then the payment of the benefit amount is made to the policy owner and not the veterinary practice. In either case it is expected that the benefit amount will be reimbursed by the insurance provider within around 48 hours. The insurance provider 13 is notified of payment of the gap by the policy owner or associated individual via the VPMS 14. In this example, the reimbursement by the insurance provider 13 is only triggered via communication from the VPMS 14 in the event that the gap amount is paid in full to the veterinary practice.

In another example, the method comprises the step of notifying payment of the benefit amount by the insurance provider, responsive to a determination that the benefit amount satisfies a predetermined criteria. In this example, notification of gap payment by the policy owner can therefore be bypassed. In other words, reimbursement by the insurance provider 13 is triggered if the benefit amount satisfies a predetermined criteria, regardless of any communication from the VPMS 14 that the gap amount is paid to the veterinary practice. The determination may be made at or by the software platform 10 or insurance provider 13, remotely from the VPMS 14. The predetermined criteria may be that the benefit amount is at lower than a threshold amount, such as $1,000. Use of a predetermined criteria to trigger the reimbursement improves efficiency of claim processing by automatically bypassing notification steps. Such automation may shorten claim processing time by reducing either or both machine and human intervention. Further, use of a threshold amount as the predetermined criteria assists in financial risk reduction, by providing downside protection.

The predetermined criteria may be reconfigurable, such as having a reconfigurable threshold amount. For example, all the veterinary practices and/or policy owners may be associated with a default of initial threshold amount, such as $1,000. To further adjust financial risks and/or claim processing efficiencies, the threshold amount may be increased or decreased from $1,000, responsive to one or more predetermined events. A predetermined event may include one or more successful claims associated with a particular veterinary practice and/or a particular policy owner, in which case the threshold amount may be increased accordingly due to a satisfactory claim history. Alternatively or additionally, a predetermined event may include one or more unsuccessful or problematic claims associated with a particular veterinary practice and/or a particular policy owner, in which case the threshold amount may be decreased accordingly due to one or more claim rejections. Further, the method may include the step of auditing a sample of benefit payments as a result of the determination that the benefit amount satisfies a predetermined criteria. For example, a portion (e.g. 10%) of all veterinary practices are randomly selected regularly (e.g. each month) to verify receipt of the corresponding gap payments. The audit may include requesting and/or receiving evidence or proof (e.g. a screenshot of the veterinary practice's software and associated user interface) that the corresponding gap payments have been received from the respective policy owners. A successful audit, such as the satisfactory presentation of such evidence, may be a predetermined event to increase the threshold amount for that particular veterinary practice. A failed audit, such as the absence of such evidence, may be a predetermined event to decrease the threshold amount for that particular veterinary practice.

It should be noted that, in addition to notifying payment of the benefit amount by the insurance provider responsive to the predetermined criteria, the method may still comprise notifying payment of the benefit amount by the insurance provider responsive to receipt of notification of payment of a remaining or gap amount. In other words, reimbursement can be triggered by both of a predetermined criteria (e.g. low enough benefit amount) and a communication from the VPMS 14 that the gap amount is paid to the veterinary practice. Such a dual-stream reimbursement arrangement facilitates quicker claim processing for smaller claims, while reserving human or machine resources to process larger claim amounts.

If the policy owner for whatever reason pays in excess of the remaining or gap payment, the method via the software platform 10 implements the following steps:
1. the excess payment is assessed at the UPM 13 and deducted from the benefit amount to otherwise be reimbursed to the veterinary practice 14;
2. the UPM 13 repays the policy holder the excess payment.

Although not fully illustrated, the method further comprises the step of reading the claim data to identify or flag the insurance claim as a benefit claim where the veterinary practice is potentially eligible for receipt of a benefit amount paid by the insurance provider. This may be effected by the veterinary practice categorising or flagging the insurance claim as a claim for which the policy owner may have elected to pay the gap only. In the event the insurance claim is identified as a benefit or gap only claim, the method further comprises the step of prompting the insurance provider to promptly assess the insurance claim following its receipt and processing. In this embodiment the insurance provider or staff is prompted at 30 by a telecommunication alert notifying them of the receipt of the insurance claim and in anticipation of electronic submission of the integrated claim workflow associated with the insurance claim.

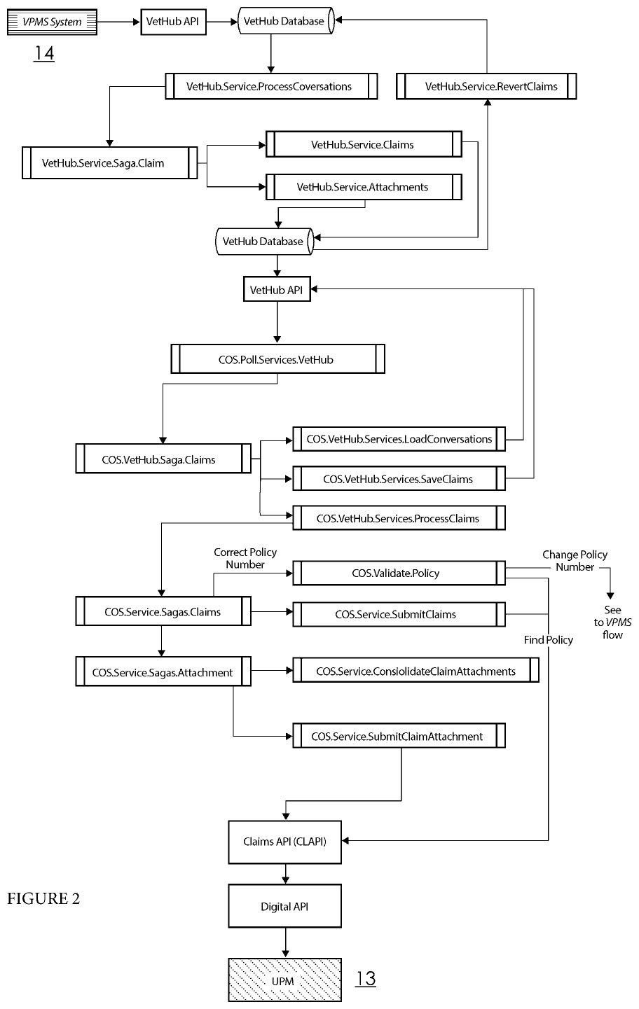

FIG. 2 illustrates in more detail part of the software platform of this embodiment of the invention built in the microservice architecture. Implementation of the preferred methodology uses the Saga pattern to manage workflows within the illustrated software platform. The microservices of this embodiment and of relevance to FIG. 2 are listed below:

| | Type | |
|---|---|---|
| | Windows/ | |
| Service Name | NServiceBus | Description |
| VetHub.Service.ProcessCoversations | Windows | Service to read all new conversations submitted |
| | | to VetHub API and push them to VetHub Saga. |
| VetHub.Service.Saga.Claim | NServiceBus | Saga for VetHub inbound claims workflow. |
| VetHub.Service.Claims | NServiceBus | Service to process conversations and mark them |
| | | ready for download |
| VetHub.Service.Attachments | NServiceBus | Generates attachments for RxWorks claims |
| VetHub.Service.RevertClaims | NServiceBus | Re-process failed conversations. |
| COS.Poll.Services.VetHub | Windows | Poll for conversation which are ready to download |
| | | and push them to COS.VetHub.Saga.Claims queue |
| COS.VetHub.Saga.Claims | NServiceBus | Saga for COS inbound claims workflow from |
| | | VetHub. |
| COS.VetHub.Services.LoadConversations | NServiceBus | Service to load all conversations to COS VetHub |
| | | Conversation table |
| COS.VetHub.Services.SaveClaims | NServiceBus | Downloads Claim and Attachments from VetHub |
| COS.VetHub.Services.ProcessClaims | NServiceBus | Prepares claim object and submits to COS Claim |
| | | Saga |
| COS.Service.Sagas.Claims | NServiceBus | Saga for COS inbound claims workflow into |
| | | Insurance system via Claims API. |
| COS.Service.ValidatePolicy | NServiceBus | Validate Policy number against Insurance records. |
| COS.Service.SubmitClaims | NServiceBus | Submit claim to Insurance system. |
| COS.Service.Sagas.Attachment | NServiceBus | Saga for managing attachments into insurance |
| | | system. |
| COS.Service.ConsolidateClaimAttachments | NServiceBus | Consolidate all attachments into single PDF |
| | | attachment. |
| COS.Service.SubmitClaimAttachment | NServiceBus | Submit claim attachment to insurance system. |

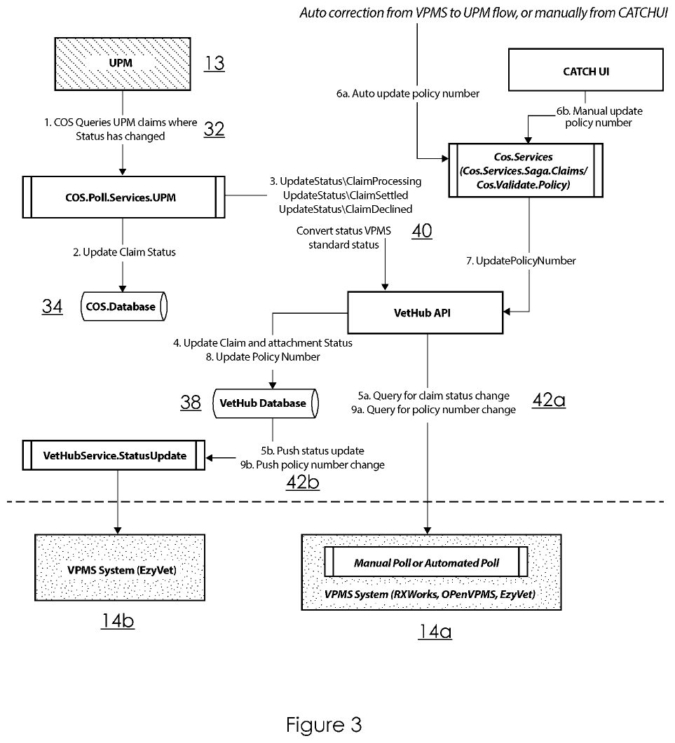

FIG. 3 illustrates remainder of the software platform dedicated largely to functionality associated with updating the status of the insurance claim and extracting the associated benefit amount.

In this example the step of receiving notification of the benefit amount from the insurance provider 13 involves electronically querying the insurance provider 13 in relation to a benefit amount being payable for the treatment of the animal at the veterinary practice such as 14a or 14b. The insurance provider or underwriting platform management (UPM) 13 is periodically polled in relation to the benefit amount. In the event of a change in the status of the insurance claim, the UPM at 32 responds to the periodic polling with notification of an updated status including the benefit amount.

In this embodiment the method also comprises the step of storing the notification of the benefit amount received from the insurance provider or UPM 13 in an insurance integration database at 34. The benefit amount may be entered into the insurance integration database 34 for storage via an associated insurance user interface at 36 (see FIG. 1).

Returning to FIG. 3, the method still further comprises the step of sending the notification of the benefit amount to a claim workflow database at 38. The notification of the benefit amount is in this example converted to a compatible data format at 40 capable of being read by the veterinary practice 14a/b. In this case the compatible data format is based on a canonical data model consistent with claim data received from the veterinary practice, or the veterinary practice management system (VPMS) 14a/b. The compatible data format is typically an industry standard data format such as VetXML.

In this embodiment the step of electronically informing the veterinary practice 14a/b of the benefit amount involves prompted delivery of the benefit amount at either 42a or 42b to the veterinary practice in the compatible data format. This prompted delivery of the updated status including the benefit amount is either i) in response to periodic polling at 42a from the veterinary practice 14a or ii) resulting from periodic pushing at 42b of the benefit amount to the veterinary practice 14b. The benefit amount is thus reported to the veterinary practice in substantially real time. This allows the policy owner or associated individual to attend to payment of the remaining or gap amount at the time of attending the veterinary practice for the treatment. It is expected that substantially real time reporting of the benefit amount to the veterinary practice and their determination of the remaining or gap payment (based on the invoiced amount) will be effected in less than around 10 minutes meaning the policy owner/customer is not waiting an extended period to settle their invoice.

In the event the insurance claim is declined by the UPM 13 this is communication together with notification of an updated status to VPMS 14a/b. The veterinary practice will then be required to collect the full invoiced amount from the policy holder/customer. The VPM 13 will not make any benefit amount payments to either the customer or the veterinary practice. If the insurance claim for whatever reason cannot be processed by the software platform 10 or the UPM 13, the veterinary practice will be notified of this failure either by telephone from insurance staff or via the VPMS 14a/b, possibly in conjunction with notification of an updated status from the UPM 13. Once again the veterinary practice will then be required to collect the full invoiced amount from the policy holder/customer.

The microservices of this embodiment and of relevance to FIG. 3 are listed below:

| | Type | |
|---|---|---|
| | Windows/ | |
| Service Name | NServiceBus | Description |
| COS.Poll.Ser- | Windows | Poll for conversation which are ready |
| vices.VetHub | | to download and push them to |
| | | COS.VetHub.Saga.Claims queue |
| COS.Poll.Ser- | Windows | Periodic poll query to UPM insurance |
| vices.UPM | | system for status changes. |

As best seen in FIG. 1, it is to be understood that the insurance provider of the invention in the context of the preferred embodiment includes the insurance underwriter together with the associated UPM 13, the insurance user interface (UI) 36 and the insurance staff 45 for claims assessment and management. The claim workflow of the invention in the context of the preferred embodiment includes the API components 16 and the associated claim workflow database 38. The integrated claim workflow of the invention in the context of the preferred embodiment includes the COS 22 and the associated insurance integration database 34.

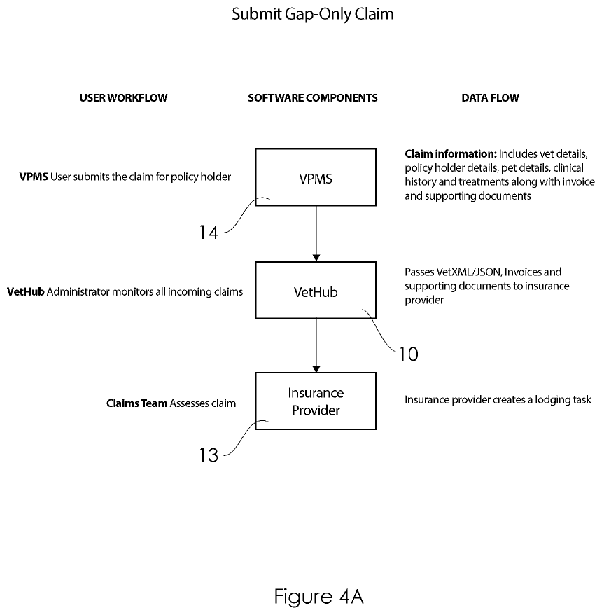

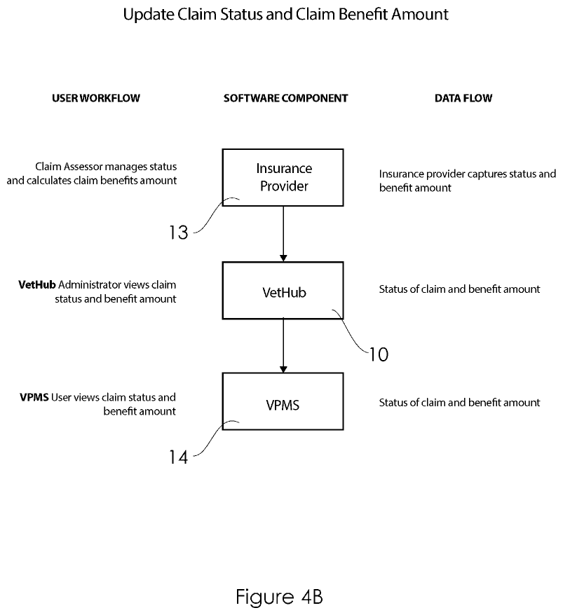

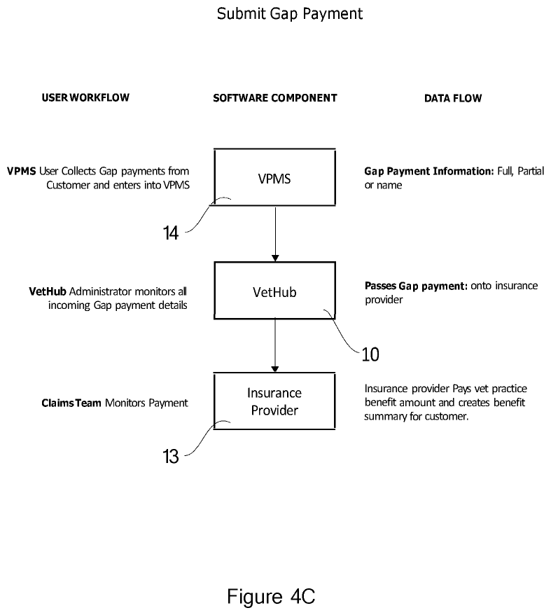

FIGS. 4A to 4C are high level illustrations of the software components relevant to the computer system of another aspect of the invention for managing an animal insurance claim. In this example and as seen in FIG. 4A, the relevant workflows in submission of the insurance claim are as follows:
1. staff at the veterinary practice 14 submit an insurance claim under an insurance policy for treatment of an animal at the veterinary practice 14;
2. the software platform 10 receives and processes claim data associated with the insurance claim;
3. the insurance provider 13 assesses the insurance claim under the insurance policy for which the treatment of the animal is provided at the veterinary practice 14.

This illustration also details examples of user workflows and data flow in the course of submission and processing of the claim. It will be appreciated that the specific information and data type may vary from the examples listed and still remain within the scope of the invention. The processing of received claim data processed by the software platform 10 may include augmenting the received claim data to arrive at the processed claim data. In one example, augmentation of the received claim data includes inserting additional data, derived from the received claim data, to the received claim data to arrive at the processed claim data. The derived data may be derived in accordance with predetermined data format, such as industry accepted standards. Further, such augmentation may include retaining all received claim data in the processed claim data, such as that no received claim data is removed. In other words, processed claim data to be sent to and assessed by the insurance provider 13 includes both the received claim data and the derived data.

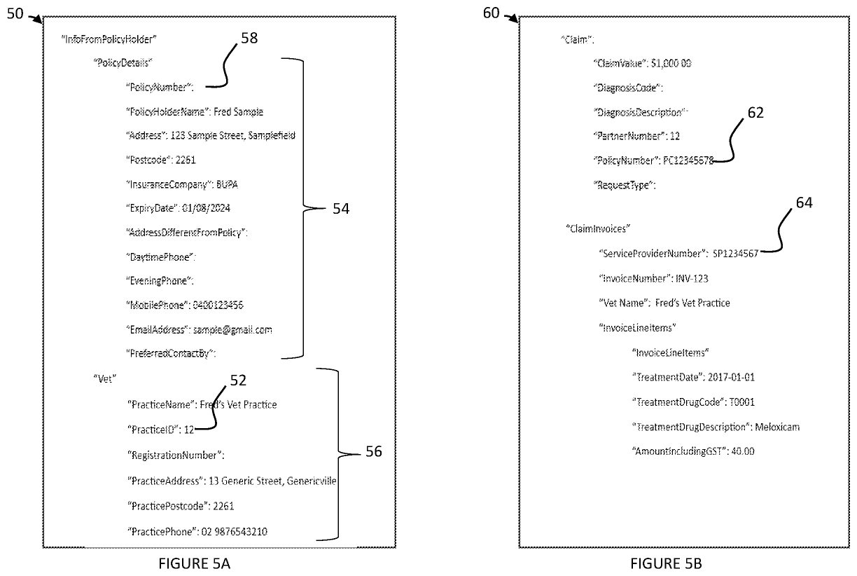

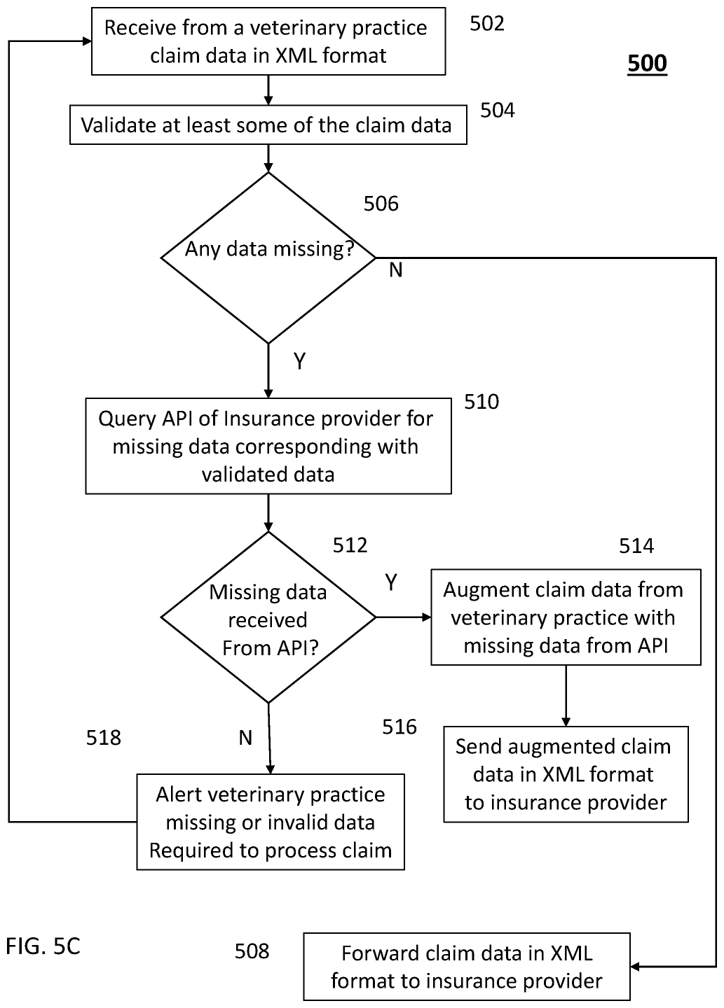

FIGS. 5A and 5B show an example of claim data augmentation. FIG. 5A illustrates, at least partially, claim data in XML format 50 received by software platform 10 from veterinary practice 14. FIG. 5B illustrates, at least partially, claim data in XML format 60 as a result of processing by software platform 10. It is to be understood that the processed claim data in XML format 60 includes (but for simplicity is not shown in FIG. 5B to include) the received claim data in XML format 50 in its entirety. The received claim data in XML format 50 includes pet details (not shown), claim details (not shown), veterinary practice details 56 and policy holder details 54. In the example shown, the policy number is missing. The missing policy number may be due to the Policy Number field 58 being a non-mandatory entry and thus left incomplete by operators of the veterinary practice 14 providing the claim data in XML format 50. As another example (not shown), there are cases where an invalid policy number is provided by the veterinary practice 14. The software platform 10 may be configured to verify or determine validity of at least some of the provided claim data. Where claim information is missing or determined to be invalid, the software platform 10 is configured to retrieve the missing or valid information. For example, if the policy number in the Policy Number field 58 is missing or determined invalid by the software platform 10 (e.g. by making an API call to the insurance provider to check whether for a valid policy number), the software platform 10 is configured to locate a matching policy record based on associated policy holder data (e.g. by making an API call to the insurance provided to check for suburb, mobile phone number, surname etc.). Where a matching policy record is located, the software platform 10 is configured to retrieve the policy number (in this example, ‘PC12345678’) from the matching policy record. The software platform 10 is configured to augment the received claim data in XLM format 50 by adding the retrieved policy number 62 to the processed claim data in XML format 60. The processed claim data in XML format 60 is then provided to the insurance provider.

Also as shown in the example, the received claim data in XML format 50 includes a PracticeID field 52, which contains an indicator of the veterinary practice recognizable by the software platform 10. The software platform 10 is configured to identify which insurance provider to send the corresponding claim to. Once the software platform 10 identifies the associated insurance provider, the software platform 10 augment the received claim data in XML format 50 by adding the corresponding veterinary number 64 to the processed claim data in XML format 60. In this example, ‘SP1234567’ is the unique number for Fred's Vet Practice within the insurance provider system. The processed claim data in XML format 60 is then provided to the insurance provider.

The method 500 described generally above with respect to FIGS. 5A and 5B is further described in connection with FIG. 5C. As depicted in FIG. 5C, the method 500 of the software platform 10 receives claim data from a veterinary practice in XML format at step 502. At step 504, at least some of the data is validated. At step 506 a determination is made whether there is any missing data (e.g., data that the software platform is programed to understand the insurance provider will require to act on the claim). If there is no missing data, the claim is forwarded to the insurance provider at step 508. However, if the software platform 10 determines that the received data from the veterinary practice is missing certain data, the software platform queries the insurance provider API at step 510 to provide the missing data. As noted above, at least some of the data from the veterinary practice is used to identify a policy at the insurance provider corresponding to the received data from the veterinary practice. At step 512 a determination is made whether the missing or incorrect data requested at step 510 has been received. If it has been received the method progresses to step 514 where the received missing data from the insurance provider API is used to generate augmented claim data. As noted above, this missing data, when combined with the data received from the veterinary practice completes the necessary data for the insurance provider to act on the claim. At step 516, the augmented claim data is transmitted to the insurance provider in XML format for further processing in the ordinary course by the insurance provider, and as described elsewhere herein. In instances where insufficient missing or incorrect data can be received from the insurance provider API at step 512, the method may progress to step 518 and the veterinary practice is alerted to the insufficiency of the data for the processing of the claim, and thus require resubmission which when entered can be received by the software platform and the method reverts to step 502. Further, though described herein as requiring querying the insurance provider API (step 510) the missing data may also be retrieved from one or more databases associated with the software platform for augmentation of the claim data and submission to the insurance provider.

FIG. 4B illustrates workflows associated with changes in the status of the insurance claim and more particularly notification of the benefit amount where:
1. a claims assessor at the insurance provider 13 updates the status of the insurance claim, for example “processing” “failed processing” “settled” or “declined”, and if relevant their assessment of the benefit amount to be paid by the insurance provider 13;
2. the software platform 10 receives notification of the benefit amount under the insurance policy for the treatment;
3. the veterinary practice or the VPMS 14 is informed of the benefit amount, typically to be paid by the insurance provider 13 to the veterinary practice 14.

FIG. 4C illustrates workflows associated with submission of the remaining or gap payment made by the policy holder or the associated individual where:
1. staff at the veterinary practice receive the remaining or gap payment from the customer and enter it into the VPMS 14;
2. the software platform 10 receives notification of the gap payment under the insurance policy for the treatment;
3. the insurance provider 13 is informed of the gap payment triggering payment of the benefit amount and prompting dispatch of a benefit summary to the policy holder.

It will be understood that the methodology adopted by the software platform 10 of the preceding aspect of the invention is enabled by the various components of the computer system of this aspect for managing animal insurance claims. These components may be in the form of either software or hardware associated with the software platform 10.

FIG. 1 depicts the specific steps involved in managing an insurance claim in the form of the gap only claim for payment of a benefit amount by the insurance provider. It will be appreciated that the particular order and detail for each of the steps may vary whilst remaining within the broad scope of the invention. Following the numbering of the annotated steps in the flow diagram, the method in this embodiment functions as follows:
1a. to 1c. VPMS calls a vet hub API of the platform to submit the claim in an industry standard canonical model. Vet hub stores the claim, and all attachments in the canonical model format at a claim workflow database.
2. COS polls the vet hub API to query for new claims submitted, and retrieves them to COS from the claim workflow database.
2b. COS sends a notification (phone call) to insurance staff to alert the insurance provider of the gap only claim.
3a. COS validates the policy number of the claim against corresponding UPM data, and corrects an invalid policy number using other fields (name, address, etc).
3b. COS submits the claim to the UPM under a high priority queue.
4. Insurance staff process the claim from the high priority queue.
5. COS periodically polls the UPM to see if the claim status has changed, and if so, the updated status including the benefit amount is retrieved from the UPM and automatically saved into COS.
6a & 6b. Alternatively, once the UPM calculates the benefit amount, the benefit amount is entered into an insurance provider UI and saved into COS.
7. COS sends the updated status and benefit amount to the vet hub API, which is saved in the canonical model format in the claim workflow database.
8a & 8b. VPMS periodically polls the vet hub API querying for claim status changes and benefit amount and/or policy number changes. If changed, it will update the status and communicate the benefit amount to the veterinary practice or VPMS.
8c. Alternatively, the vet hub API will call VPMS to push claim status changes and the benefit amount and/or policy number changes to it. If changed, it will update the status and communicate the benefit amount to the vet practice or VPMS.
9a. to 9c. The veterinary practice advises the policy owner or customer of the benefit amount and the gap. The customer pays the gap only, and that value is sent back to the vet hub API. Where the benefit amount satisfies a predetermined criteria (e.g. lower than a threshold amount), steps 9 to 13 proceed regardless of payment of the gap.
10. The vet hub API pushes any new gap payments to COS.
11. COS updates the claim to the UPM with the gap payment amount. The UPM marks the gap only claim as complete. Alternatively COS informs the insurance staff of the new gap payment by the customer and the gap payment amount is manually entered by the staff at the Catch UI.
12. The UPM reimburses the veterinary practice the benefit amount, typically via bank transfer.
13. The UPM sends a benefit statement to the customer (email or post).

Now that a preferred embodiment of a computer-implemented method of managing an insurance claim has been described it will be apparent to those skilled in the art that it has the following advantages:
1. the method and associated computer system enable substantially real time submission and assessment of an insurance claim for payment of the remaining or gap amount at the time of attending the veterinary practice for the required treatment;
2. the method and system allow a veterinary practice with their existing software to communicate directly with the insurance provider without for example having to rely upon a client/browser application;
3. the method and system of the software platform in adopting an industry standard data model integrate with the veterinary practice and in particular the VPMS, typically using an industry standard data format;
4. the method and system are configured for integration with various insurance providers with the insurance integration component of the software platform being tailored for compatibility with the insurance provider or UPM;
5. the method and system enable relatively quick reimbursement of the veterinary practice of the benefit amount by the insurance provider following or bypassing notification of payment of the remaining or gap only amount by the policy owner;
6. the method and system significantly reduce the time and effort otherwise required by the policy owner in manually submitting an insurance claim together with supporting documents for reimbursement of the benefit amount payable by the insurance provider for the claim.

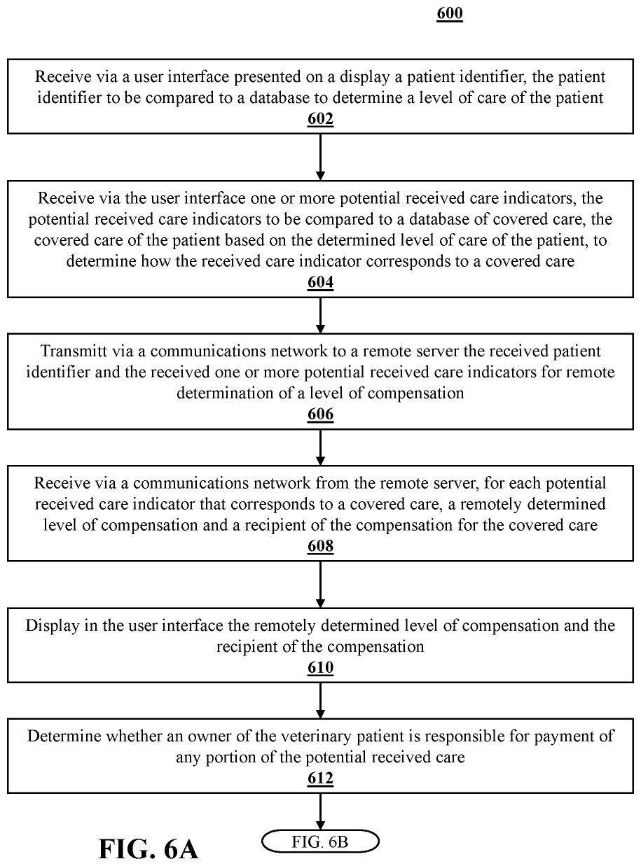

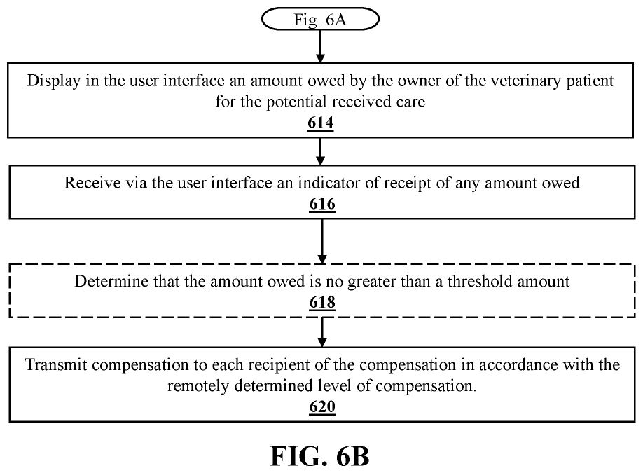

FIGS. 6A and 6B depict a method 600 of one aspect of the systems and processes described herein above. In connection with the method at step 602 a patient identifier (e.g., a policy number, policy holder's name, etc.) is entered into the VPMS (e.g., 14a) via a user interface displayed on a computing device. At step 604 potential therapies or care to be provided to the patient (i.e., a diagnosis code or proposed treatment) are received by the VPMS via the user interface. For example, the VPMS may display a variety of fields on the user interface requiring the entry of the requested data. At step 606 the patient identifier and potential therapy are transmitted to a remote server (e.g., software platform 10 and/or UPM 13) for determination of a level of compensation. At step 608 the VPMS receives either directly or indirectly (e.g., via the software platform 10 or UPM 13) from the remote server a determination whether the potential therapies are care that is covered by the policy, a level of compensation from the insurance carrier for each potential therapy, and a recipient of the compensation for the covered care. At step 610 the information received in step 608 is displayed on a user interface (e.g., on a display of a computing device). At step 612, a determination is made whether the owner of the veterinary patient is responsible for payment of any portion of the potential care and at step 614 the amounted owed is displayed on the user interface. At step 616 an indication of receipt of the amount owed is received by the software platform 10 or optionally at step 618 a determination can be made that the amount owed is less than a threshold amount. At step 620 the insurance carrier transmits the compensation to each of the identified recipients identified in step 608.

Those skilled in the art will appreciate that the invention as described herein is susceptible to variations and modification other than those specifically described. Although the invention as broadly described is directed to notification of the benefit amount, the method may also extend to notification of the remaining or gap amount derived from the benefit amount, typically by deducting the benefit amount from the invoiced amount. The workflows, routines or other functionality of the software platform may depart from the preferred embodiment provided it remains broadly within the scope of the broadest aspects of the invention. The standard data format of the claimed data ingested by the software platform may depart from the canonical model provided the VPMS is compatible with the vet hub API or associated claim workflow database. The retrieval of workflows or other data from either the claim workflow database or the insurance integration database may depart from the poll/respond or push models implemented by the API and COS components of the preferred software platform.

All such variations and modifications are to be considered within the scope of the present invention the nature of which is to be determined from the foregoing description.

1. A veterinary system comprising:
a computing device including a display, a processor and a memory, the memory storing instructions that when executed by the processor perform the steps of:
receiving via a user interface presented on the display a patient identifier, the patient identifier to be compared to a database to determine a level of care of the patient;
receiving via the user interface one or more potential received care indicators, the potential received care indicators to be compared to a database of covered care, a covered care of the patient based on the determined level of care of the patient, to determine how the received care indicator corresponds to a covered care;
transmitting via a communications network to a remote server the received patient identifier and the received one or more potential received care indicators for remote determination of a level of compensation;
receiving via the communications network from the remote server, for each potential received care indicator that corresponds to a covered care, a remotely determined level of compensation and a recipient of the compensation for the covered care;
displaying in the user interface the remotely determined level of compensation and the recipient of the compensation;
transmitting compensation to each recipient of the compensation in accordance with their remotely determined level of compensation; and
determining whether a policy owner is responsible for payment of any portion of the potential received care;
displaying in the user interface an amount owed by the policy owner for the potential received care; and
receiving via the user interface an indicator of receipt of any amount owed, wherein the transmission of the compensation is performed responsive to:
receipt of the indicator of receipt of the amount owed; or
a determination that the amount owed is no greater than a threshold amount, regardless of the receipt of the indicator of receipt of the amount owed.

2. A veterinary system of claim 1, wherein:
the user interface is a first user interface of a plurality of user interfaces, the first user interface being configured to correspond to a first veterinary software used at a first veterinary practice;
the first interface is different from a second user interface of the plurality of user interfaces, the second user interface being configured to correspond to a second veterinary software, different from the first veterinary software, used at a second veterinary practice, different from the first veterinary practice; and
the first and second user interfaces are both configured to receive a predetermined set of common indicators for transmission to the remote server.

3. A veterinary system of claim 1, wherein the memory includes further instructions that when executed by the processor perform the further steps of:
receiving reconciliation data associated with the compensation; and
displaying in the user interface a reconciliation indicator corresponding to the reconciliation data.

4. A veterinary system of claim 3, wherein the reconciliation data include any one or more of, for each one of a plurality of compensation:
a fee for the transmission of the compensation;
an effective compensation based on the compensation reduced by the fee;
a date of the transmission of the compensation; and
a transmission reference for the transmission of the compensation.

5. A veterinary system of claim 1, wherein the determination of whether the amount owed is no greater than the threshold amount is made by the remote server.
